(19) United States
(12) Patent Application Publication
(10) Pub. No.: US 2008/0294061 A1
(43) Pub. Date: Nov. 27, 2008

(54) Health care gaming device and method using the same

(76) Inventors: Shiow-Chen Wang, Taipei (TW); Yin-Han Li, Kaohsiung (TW)

Correspondence Address: MARTINE PENILLA & GENCARELLA, LLP, 710 LAKEWAY DRIVE, SUITE 200, SUNNYVALE, CA, 94085, US

(21) Appl. No.: 11/894,429

(22) Filed: Aug. 20, 2007

(30) Foreign Application Priority Data
May 25, 2007 (TW) 96118783

(51) Int. Cl. A61B 5/091; A63F 9/24

(52) U.S. Cl. 600/538; 463/1

(57) ABSTRACT

A health care gaming device includes: a case, which can be placed on a platform or be handheld by an user; at least one communicating tube provided in the case; an inflow/outflow unit connected to the communicating tube, for the user to exhale and inhale gas; at least one sensor connected to the communicating tube, for sensing gas inflow and outflow and detecting gas flow; a processing unit connected to the sensor, for measuring pulmonary function and executing games; a display unit connected to the processing unit; and a keying unit connected to the processing unit, wherein the sensor generates a sensed deformation due to gas flowing in the communicating tube; the processing unit analyzes the sensed deformation and converts the analytical result to measurement data for pulmonary function or uses the analytical result as game data for proceeding games; and the display unit displays the measurement data and game pictures. The invention also discloses a method using such health care gaming device.

### BACKGROUND OF THE INVENTION

a) Field of the Invention

The invention relates to a gaming device and a method using the same. More particularly, the invention relates to a health care gaming device which uses sensors to measure pulmonary function and games to train lungs and a method using the same.

b) Description of the Related Art

Along with the advancement of information industries and increasing needs in consumer entertainment, gaming products and accessories are flourishing, among which video game consoles like Xbox and Playstation are popular to people of different classes, and ways to control and play games are becoming more human-friendly. Conventional control methods use buttons to control an on-screen target in response to game scenario, and a user can enjoy the game in the playing process. However, in proceeding the game, the user is often kept in one posture and mainly controls the on-screen target with hands only. This will induce harms to body functions after a long duration of playing, which is a problem that cannot be overlooked.

In recent years, people are more concerned about medical and health issues, especially physical and mental illness testing. Therefore, consumer electronic medical or health products are more common in households because they are portable, easily operable, suitable for personal or family use, and not limited by time or place, which allows users to know their current health condition and achieves prevention and health purposes.

Physiological functions of lungs are primarily determined based on readings of many pulmonary function tests, including: forced vital capacity (FVC), forced expiratory volume in the first second (FEV1), total forced expiratory volume, . . . etc. Most people use pulmonary function testers to obtain the readings that are generated to help them understand personal pulmonary condition or to be provided to doctors for diagnosis. In addition, asthma patients can also use pulmonary function testers to check whether their respiration is normal, so as to prevent sudden occurrence of the illness.

Some pulmonary function testers are used for training lungs, whose purpose is to prevent complications of atelectasis after surgery by observing airflow of deep breathes in normal condition repeated for a period of time to simulate or restitute the lung pressure or vital capacity of a healthy state. Since these respiratory trainings only provides pure data observation, and the time of each respiratory training session must be consistent to achieve expected result, there may be incomplete breathing in the process that the user is not aware of, or at times the training duration is prolonged or the expected result is not achieved because the user has lost patience. Hence, the training would lose its meaning.

There are currently two types of pulmonary function testers—floating ball type and fan type. The floating ball type tester uses the principle of a lightweight sphere being lifted by wind force. When a sphere is placed in a confined space and is floating due to air filling therein, the changes to the floating sphere is recorded and converted to obtain data relating to vital capacity. However, for the floating ball type tester, a sphere would flap irregularly when force is applied unevenly, and so in consideration for accuracy, the required structural precision and cost rise in proportion. Hence, data obtained by floating ball type testers is primary for reference only.

The fan type tester uses a light sensor and fan blades to obtain data. We explain how a fan type tester operates in the following. After air is blown into the tester through a blowing tube, the fan blades rotate due to airflow, then the fan blades and the spaces between fan blades in turn pass by the sensing point of the light sensor for the light sensor to detect the number of high-low potentials which are then transmitted by electronic circuit to microprocessor for analysis and comparison. Subsequently, the reference data for pulmonary function is converted from preset values according to the level of air blown. However, the structure of this kind of testers is more complicated, making it harder to disassemble and clean and having higher cost, and therefore the price and usage convenience of fan type testers are not satisfactory.

Therefore, accurately measuring pulmonary functions with lower cost and increasing lung training efficiency with intriguing routine are the objects of the invention.

### BRIEF SUMMARY OF THE INVENTION

An object of the invention is to provide a health care gaming device that allows a user to measure his pulmonary function and use games for lung training, and a method using the same.

A health care gaming device of the invention includes: a case, which can be placed on a platform or handheld by a user; at least one communicating tube provided in the case; an inflow/outflow unit connected to the communicating tube, for the user to exhale and inhale gas; at least one sensor connected to the communicating tube, for sensing gas inflow/outflow and detecting gas flow; a processing unit provided in the case and connected to the sensor, for measuring pulmonary function and executing games; a display unit connected to the processing unit; and a keying unit connected to the processing unit, for the user to select functions of the gaming device, set game parameters, and play games; wherein the sensor generates a sensed deformation due to gas flowing in the communicating tube, and the processing unit analyzes the sensed deformation and converts the analytical result to measurement data for pulmonary function or uses the analytical result as game data for proceeding games, while the display unit displays the measurement data and the game pictures. The gaming device further includes a hand-pressing device for game operation and hand exercise.

The invention also discloses a method using a health care gaming device, wherein the health care gaming device includes: an inflow/outflow unit, at least one sensor, a processing unit, and a display unit. The method includes steps of: a user selecting measuring function or training function of the gaming device; the user exhaling and inhaling gas by using the inflow/outflow unit; the sensor sensing gas inflow/outflow and detecting gas flow; the processing unit analyzing the gas inflow/outflow; when the user selects the training function, the processing unit using the analytical result as game data and the display unit displaying the game pictures; and when the user selects the measuring function, the processing unit converts the analytical result to measurement data for pulmonary function and the display unit displaying such data.

The health care gaming device according to the invention is a gaming device that is capable of accurately measuring pulmonary function and operable by respiration. Besides, the gaming device can be utilized with peripheral units. With the gaming device, not only can a user understand his pulmonary condition, the user can also conduct respiration in response to the game scenario, which helps to increase or restitute lung pressure or inspiratory volume. Moreover, the gaming device can also include a hand operation mode in which a hand-pressing device for massaging hand muscles is used, inputting a different game controlling mode to an original operation mode, and together with the variations in game content, the user can have fun with the games while achieving the purpose of health care.

### BRIEF DESCRIPTION OF THE DRAWINGS

FIG. 1 is a block diagram illustrating the main structure of a health care gaming device of the invention.

FIG. 2 is an exploded view of a health care gaming device according to an embodiment of the invention.

FIG. 3 is a cross-sectional view of a health care gaming device according to an embodiment of the invention.

FIG. 4 is a front view of a health care gaming device according to another embodiment of the invention.

FIG. 5 is a flow chart illustrating a method using a health care gaming device of the invention.

FIG. 6 is a block schematic diagram illustrating an execution of game content.

FIG. 7 is a schematic diagram illustrating a flow mechanism of the invention.

FIG. 8A is a schematic diagram illustrating the internal connection in a health care gaming device according to an embodiment of the invention.

FIG. 8B is a schematic diagram illustrating another internal connection in a health care gaming device according to an embodiment of the invention.

### DETAILED DESCRIPTION OF THE INVENTION

The health care gaming device and the method using the same according to preferred embodiments of the invention will be described in detail with reference to the drawings, in which like reference numerals denote like elements.

A health care gaming device of the invention has a main structure as shown in FIG. 1, which includes: means for gas inflow/outflow 12, a sensing element 13 connected to the means for gas inflow/outflow 12, a processing unit 14 connected to the sensing element 13, a display unit 15 connected to the processing unit 14, and a keying unit 16 connected to the processing unit 14. The means for gas inflow/outflow 12 is composed of an inflow/outflow unit (not illustrated in FIG. 1) and at least one communicating tube (not illustrated in FIG. 1), wherein a user uses the inflow/outflow unit to exhale and inhale gas and the gas flows through the communicating tube and reaches/advances to the sensing element 13. The sensing element 13 is used to sense the inflow/outflow of gas breathed by the user and other gases and detect gas flow; a sensed deformation is generated within the sensing element 13 due to gas flow and is conveyed to the processing unit 14. The processing unit 14 is used to measure pulmonary function and execute games by analyzing the sensed deformation of the sensing element 13 and, converting the analytical result to measurement data for pulmonary function or using the analytical result as game data for proceeding games, depending on the gaming device function which the user has selected. The measurement data of pulmonary function includes forced vital capacity (FVC), forced expiratory volume in the first second (FEV1), or total forced expiratory volume. The display unit 15 is used to display the measurement data and game pictures, and the keying unit 16 is provided for the user to select the gaming device functions, set game parameters, and control/operate/play games. The health care gaming device of the invention can further include a hand-pressing device 18 connected to the communicating tube of the means for gas inflow/outflow 12, wherein the hand-pressing device 18 is a hollow compressible container and is for game operation and hand exercise.

The health care gaming device according to a preferred embodiment of the invention has the following characteristics: 1) the inflow/outflow unit of the means for gas inflow/outflow 12 is a detachable conduit, which is easy to store and clean; 2) the sensing element 13 is disposed inside the communicating tube of the means for gas inflow/outflow 12 with its sensing point positioned in front of the outlet of the inflow/outflow conduit, facing the center point of the conduit outlet. Such design is to ensure gas flowing through the inflow/outflow conduit would directly advance to the sensing point, resulting in a deformation in the sensing element 13. The deformation is transformed into a potential change. The processing unit 14 then processes the potential change and accurately calculates data for pulmonary function. The signal value received by the processing unit 14 is a potential signal that is transformed from the external physical changes of the sensing element 13. The processing unit 14 magnifies and filters the potential signal before performing an analog-to-digital conversion thereto. The potential signal is converted from an analog signal to a digital signal, from which the processing unit 14 obtains set readings, for example, the maximum value, total value, etc, and these set readings are displayed by the display unit 15. The changes sensed by the sensing element 13, or the changes in digital signals which are converted into movement values in the game, can be seen from the displayed result.

While using the game function, the preferred embodiment is characterized in that the display unit 15 displays the game content and game pictures, and through the tempo changes in the game scenario, the user uses the means for gas inflow/outflow 12 to control on-screen targets, making breathing a cyclic control.

According to an embodiment of the gaming device of the invention, the principles used in the conversion of gas flow to potential signals are:

1) relationship between flow Q and pressure P (Bernoulli Equation);

2) relationship between pressure P and force F (F=PA, A being the cross-sectional area); and

3) relationship between force F and potential signals.

The variation relationship of the gas flow and the potential can be obtained by utilizing these equations. As illustrated in FIG. 7, a conduit 71 of the inflow/outflow unit and a sensing element 72 are disposed inside a communicating tube 73, for enhancing the structural stability of the combination of the means for gas inflow/outflow and the sensing element and, limiting the flowing space for gas, the conduit 71 is detachably disposed in the communicating tube 73 whereas the sensing element 72 is fixedly disposed in the communicating tube 73. The conduit 71 has a converging outlet 74, in which gas enters the conduit 71 and at the converging outlet 74, because of the smaller tube circumference, exits at a faster flow speed, and the gas flowing out directly advances to the sensing element 72 and leaves from the outlet of the communicating tube 73 through two sides thereof.

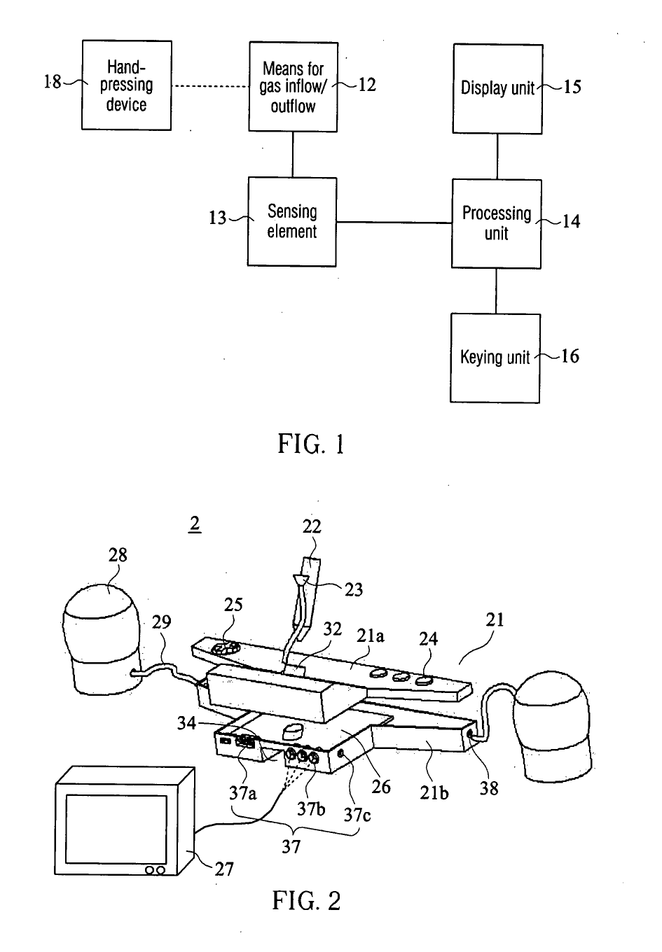

Referring to FIG. 2, a health care gaming device 2 according to an embodiment of the invention includes: a main body 21 which is easy to hold, composed of an upper casing 21a having a conduit socket 32 and a lower base 21b that can be placed on a table or a platform; a detachable blowing device 22; a detachable inhalation device 23; buttons 24 and an arrow keypad 25, disposed on the upper casing 21a; an electronic printed circuit board (PCB) 26 disposed in the main body 21; a television 27 connected to the PCB 26; and a pair of pneumatic, hallow-spherical handles, which are connected to the main body 21 by flexible tubes 29.

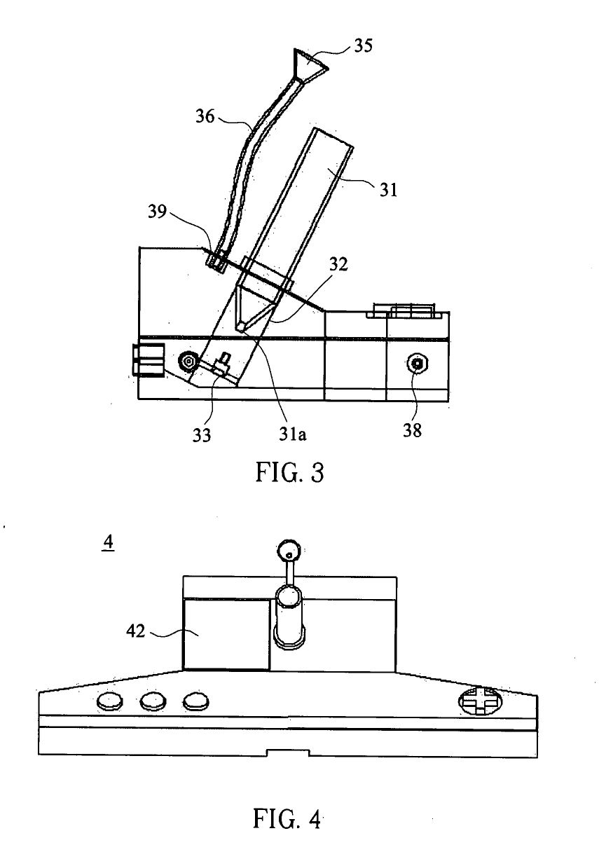

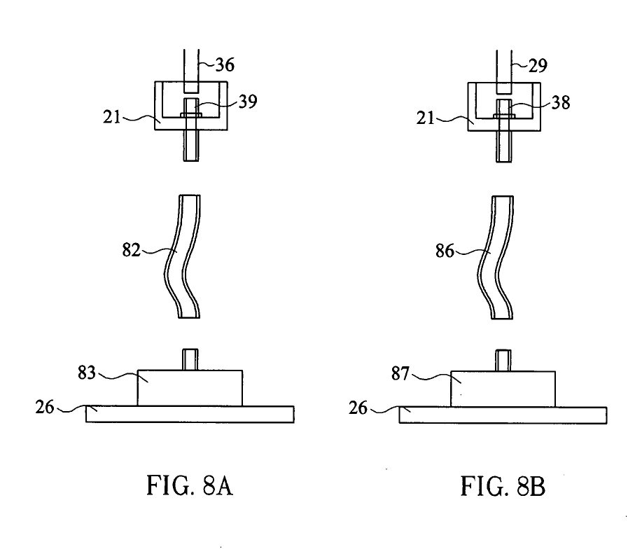

The blowing device 22 and the inhalation device 23 constitute the aforementioned inflow/outflow unit. As shown in FIG. 3, the blowing device 22 is a detachable blowing conduit 31 having a converging outlet 31a. The outlet 31a of the blowing device 22 is inserted into the conduit socket 32 which keeps the blowing device 22 in position. Gas flows out of the outlet 31a, passes an elastic sensor 33 that senses gas flow and wherein its sensing point is positioned in front of and facing the center point of the outlet 31a, and exits via a gas flow guide 34 (see FIG. 2), forming a blowing mechanism. In reference to FIG. 7, the conduit socket 32 functions similarly to the communicating tube 73, and the blowing conduit 31 functions similarly to the conduit 71. The inhalation device 23 is composed of a diverging outlet 35 and a flexible conduit 36. As shown in FIG. 8A, the flexible conduit 36 of the inhalation device 23 is connected to a gas intake 39 provided on the upper casing 21a, so as to be in connection with a communicating tube 82 provided in the main body 21. Gas inhaled by the user forms a pulling force in the conduit 36 and the communicating tube 82 and is sensed by a sensor 83 connected with the communicating tube 82, forming an inhalation mechanism.

The pair of hollow spherical handles 28 and the flexible tubes 29 constitute the aforementioned hand-pressing device 18. As shown in FIG. 8B, one end of each tube 29 connects to one of the hollow spherical handles 28 while the other end connects to one of the two vents 38 provided on the two sides of the main body 21, so as to be in connection with a communicating tube 86 provided in the main body 21. Force applied by the user's hand, either by gripping, pressing, or squeezing the handles, is conveyed to the inside of the main body 21 and a sensor 87 connected to the communicating tube 86 senses the gas flow. The surface of the spherical handles 28 has a plurality of round protrusions evenly distributed on places where the hands come in contact therewith.

The PCB 26 serves as the aforementioned processing unit 14. Functions of the PCB 26 include: (1) analog signal processing functions like magnifying, filtering, voltage regulation, and voltage step-down; (2) digital processing functions like those of a microprocessor; and (3) other functions like analog-to-digital conversion, timing, storage, driving games, radio frequency or other external connection. The PCB 26 also has transmit ports 37 for external connection, including a computer connecting port 37a and a television connecting port 37b, for data or game transmission, and an audio connecting port 37c for connecting to an external speaker.

The buttons 24 and the arrow keypad 25 constitute the aforementioned keying unit 16 and are separately provided on the left and right sides of the upper casing 21a, for selecting functions and operating games. The television 27, serving as the aforementioned display unit 15, is used for the user to read data relating to pulmonary functions or for showing game pictures. According to another embodiment, the health care gaming device 4 of the invention further includes a display screen 42 disposed on the surface of the upper casing, as illustrated in FIG. 4.

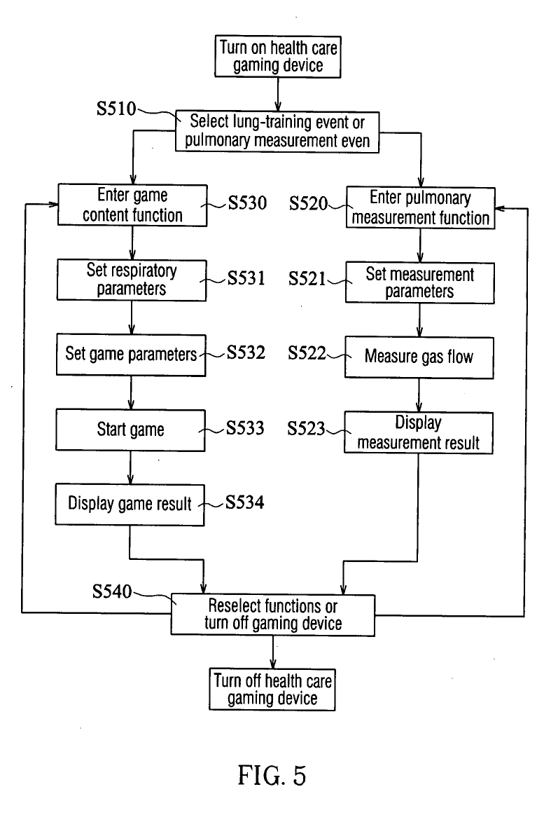

Referring to FIG. 5, the functions of the health care gaming device of the invention and the way to use these functions are described. A health care gaming device, after it is turned on, enters event selection phase (S510)—lung-training or pulmonary measurement, and a user can choose to enter the pulmonary measurement function (S520) or game content function (S530) by using the keying unit 16. In the pulmonary measurement event, the user sets measurement parameters according to the user's condition (S521), such as: time, storage setting, personal data, etc. After the parameters are set, the user exhales and inhales gas using the inflow/outflow unit and the gaming device begins to measure the gas flow (S522). The display unit 15 displays the measurement result (S523) after the measurement is finished. If the game content event is selected, the user sets respiratory parameters (S531) like inhalation time, exhalation time, breathing interval, etc. and sets game parameters according to the user's interest (S532). The game parameters include scenario, sound, and light settings. After setting the parameters, the gaming device starts to execute the game and the user proceeds to play the game using the inflow/outflow unit or keying unit 16, or optionally the hand-pressing device 18 (S533). Besides displaying the game pictures, the display unit 15 also displays the game result (S534) during each complete stage or at the end of the game.

When the selected function of the gaming device is executed and complete, the user can reselect a function of the gaming device or choose to turn it off (S540). If the user selects the pulmonary measurement function of the device, the gaming device returns to step S520, and if the user selects the lung-training function, the gaming device returns to step S530.

In the course of the pulmonary measurement function, the user blows gas into the blowing conduit 31, and the speed of gas flow increases due to a pressure generated at the converging outlet 31a, whereby the gas directly advances to the sensing point of the elastic sensor 33 after exiting the blowing conduit 31. An elastic deformation is generated at the elastic sensor 33 in response to the gas flow and the deformation is detected and converted by the PCB 26 for calculation and processing, therefore generating respiratory data of the user. The data is then displayed by the television 27, or by a computer or other device connected to the computer transmit port 37a or other transmit port (not illustrated), respectively. Alternatively, if the gaming device is provided with a display screen 42, the data can be shown by the display screen 42.

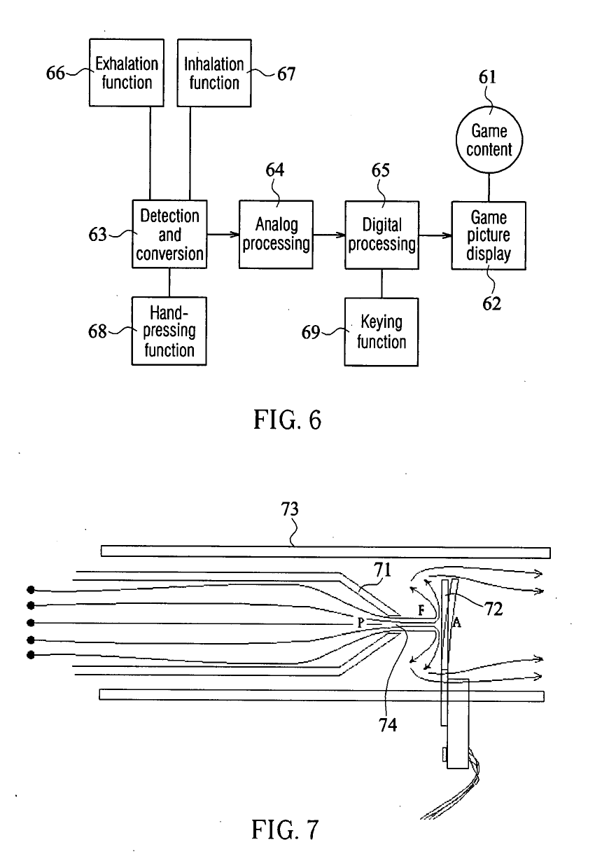

In the course of the lung-training function, the execution of game content is as shown in FIG. 6. After the game starts (S533), the game content 61 is executed and game pictures are displayed (62) by a computer, a television, or other display devices connected respectively to the computer transmit port 37a, the television transmit port 37b, or other transmit ports. Based on the on-screen target's moves requested by the displayed game content, like moving forward, backward, left, right, up, down, etc., and with the cooperation of exhalation function 66, inhalation function 67, hand-pressing function 68, and keying function 69, the game is proceeded. The exhalation function 66, inhalation function 67, and hand-pressing function 68 first undergo detection and conversion (63), converting to potential signals, and then undergo analog processing (64), and finally undergo digital processing (65). The combination of signals, which includes signals of the keying function 69 that required digital processing, creates the moves of on-screen target in the game, which is sent to the display device for game picture display (62).

The exhalation function 66 refers to the course of action in which gas is blown into the blowing conduit 31, flows out of the converging outlet 31a, advances to the elastic sensor 33, and exits via the gas flow guide 34. When the gas passes the elastic sensor 33, the elastic sensor 33 deforms due to the gas flow and the deformation is processed by the PCB 26 as described above. The inhalation function 67 refers to the course of action in which gas is inhaled from a diverging outlet 35, which generates a pulling force in the outlet 35, and the pulling force is conveyed by the flexible conduit 36 through the gas intake 39 into the inside of the main body 21. The gas is detected by the elastic sensor 83, resulting in a deformation of the elastic sensor 83, which deformation is then processed by the PCB 26 connected to the elastic sensor 83. The hand-pressing function 68 refers to the course of action in which a pressure generated by the hollow spherical handles 28 is conveyed by the tubes 29 and enters the inside of the main body 21 through the vents 38. The pressure is detected by the elastic sensor 87, resulting in a deformation of the elastic sensor 87, which deformation is then processed by the PCB 26 connected to the elastic sensor 87.

For instance, if the game is designed to control a running person by gas flow, wherein gas flow is used to represent the running speed, exhalation is used to represent running at the left side, inhalation is used to represent running at the right side, and the time for running at one side is 5 seconds. When time is up the running person is forced to run at the other side or the game is over, the user can breath in accordance with the game design to an operation cycle of 5 seconds inhalation and 5 seconds exhalation. Moreover, the game can be designed such that stones appear randomly during the game and the user needs to control the character to jump over the stones by hand force. The game is proceeded in this way and a game result is displayed (S534) at the end. The user can do respiratory and hand exercises while playing the game, which enhances the function of the game.

According to preferred embodiments of the invention, the sensing element 13 can be an elastic or piezoelectric sensing element. The processing unit 14 includes an electronic circuit, a processor, and game content, wherein the processing unit 14 can be a PCB board or a combination element that is capable of executing the functions of the abovementioned elements. The display unit 15 can be a computer, a television or a display screen. The blowing device and the inhalation device of the inflow/outflow unit can be worn on the ear or head for the convenience of the user. The gas intake 39, which plays a part in the inhalation mechanism, can be connected to a long conduit instead of the original inhalation device 23 for connecting with other breathing devices. The inhalation device 23 can also be connected to other gas generating devices, for example, an oxygen concentrator or an aroma generator.

While the invention has been described by way of example and in terms of the preferred embodiments, it is to be understood that the invention is not limited to the disclosed embodiments. To the contrary, it is intended to cover various modifications and similar arrangements as would be apparent to those skilled in the art. Therefore, the scope of the appended claims should be accorded the broadest interpretations so as to encompass all such modifications and similar arrangements.

What is claimed is:

1. A health care gaming device, which measures pulmonary function and uses games for lung training, comprising:
a case, which can be placed on a platform or handheld by an user;
at least one communicating tube provided in the case;
an inflow/outflow unit connected to the communicating tube, for the user to exhale and inhale gas;
at least one sensor connected to the communicating tube, for sensing gas inflow and outflow and detecting gas flow;
a processing unit connected to the sensor, for measuring pulmonary function and executing games;
a display unit connected to the processing unit; and
a keying unit connected to the processing unit, for the user to select functions of the gaming device, set game parameters, and play games;
wherein the sensor generates a sensed deformation due to gas flowing in the communicating tube; the processing unit analyzes the sensed deformation and converts the analytical result to measurement data for pulmonary function or uses the analytical result as game data for proceeding games; and the display unit displays the measurement data and game pictures.

2. The health care gaming device as described in claim 1, wherein the measurement data of pulmonary function includes forced vital capacity, forced expiratory volume in the first second, and total forced expiratory volume.

3. The health care gaming device as described in claim 1, wherein the inflow/outflow unit comprises a blowing device having a converging outlet.

4. The health care gaming device as described in claim 1, wherein the inflow/outflow unit comprises: an inhalation device having a diverging outlet and a flexible conduit.

5. The health care gaming device as described in claim 1, wherein the inflow/outflow unit is a detachable assembly.

6. The health care gaming device as described in claim 5, wherein the inflow/outflow unit is able to accept inflow of other gases.

7. The health care gaming device as described in claim 5, wherein the inflow/outflow unit is worn on the ear or the head of the user.

8. The health care gaming device as described in claim 1, wherein the sensor is disposed inside the communicating tube.

9. The health care gaming device as described in claim 1, wherein the sensor is an elastic or piezoelectric sensing element.

10. The health care gaming device as described in claim 1, wherein the connection between the display unit and the processing unit is wireless.

11. The health care gaming device as described in claim 1, wherein the display unit is a computer, a television or a display screen.

12. The health care gaming device as described in claim 1, wherein the game parameter setting includes: setting gas inflow/outflow data to be game data, for controlling the moves of on-screen targets in the game.

13. The health care gaming device as described in claim 1, further comprising:
a hand-pressing device, which is a hollow compressible container and connected to the communicating tube, for game operation and hand exercise.

14. The health care gaming device as described in claim 13, wherein the hand-pressing device comprises: a handling portion having a surface that is evenly distributed with round protrusions, and the surface provides massage therapy to hands that come in contact therewith.

15. A method using a health care gaming device, the health care gaming device being capable of measuring pulmonary function and using games for lung training, wherein the health care gaming device comprises: an inflow/outflow unit, a processing unit, at least one sensor, and a display unit, the method comprising:
a user selecting the measuring function or training function of the gaming device;
the user exhaling and inhaling gas by using the inflow/outflow unit;
the sensor sensing gas inflow and outflow and detecting gas flow;
the processing unit analyzing the gas flow;
when the user selects the training function, the processing unit using the analytical result as game data and the display unit displaying game pictures; and
when the user selects the measuring function, the processing unit converting the analytical result to measurement data and the display unit displaying the measurement data.

16. The method using a health care gaming device as described in claim 15, further comprising:
after the training function is selected, the user setting game parameters.

17. The method using a health care gaming device as described in claim 16, wherein the game parameter setting includes: setting gas inflow/outflow data to be game data, for controlling the moves of on-screen characters in the game.

18. The method using a health care gaming device as described in claim 15, wherein the measurement data includes: forced vital capacity, forced expiratory volume in the first second, or total forced expiratory volume.

19. The method using a health care gaming device as described in claim 15, wherein the sensor generates a sensed deformation due to the gas flow.
